(19) United States
(12) Patent Application Publication
(10) Pub. No.: US 2005/0079192 A1
(43) Pub. Date: Apr. 14, 2005

(54) Cosmetic article for treating the skin, mucous membranes, the hair or the scalp, and cosmetic treatment process

(76) Inventor: Pascal Simon, Thiais (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/900,130

(22) Filed: Jul. 28, 2004

Related U.S. Application Data
(60) Provisional application No. 60/501,025, filed on Sep. 9, 2003.

(30) Foreign Application Priority Data
Jul. 28, 2003 (FR) 03 09234

(51) Int. Cl. A61K 6/00; A61K 7/00

(52) U.S. Cl. 424/401

(57) ABSTRACT

A cosmetic article comprising a support impregnated with a cosmetic composition, wherein the support comprises: a) a contact structure configured so as to come into contact with a moistened surface onto which the cosmetic composition is to be applied, and b) an absorbent structure adjacent to the contact structure, comprising at least one water-absorbing material such that the article can absorb at least 10 times its weight of water, wherein the contact structure is configured so as: i) to be permeable to the water present on the moist surface, and ii) to act as a barrier to the at least one water-absorbing material.

This application claims benefit of U.S. Provisional Application No. 60/501,025, filed Sep. 9, 2003.

Disclosed herein is a cosmetic article, for example, for treating the skin, mucous membranes, the hair and/or the scalp. Also disclosed herein is a cosmetic treatment process using the article disclosed herein.

Cosmetic wipes generally comprise a substrate made of a material of natural or synthetic origin, which may, for example, be nonwoven, but which may also be a foam or a fabric. The substrate may be impregnated with a composition that is suited for the desired purpose, for example, cleansing or removing makeup from the skin, or alternatively for skincare. Because the wipes are disposable and may be impregnated with a sufficient amount of cleansing or treating product, they are commonly used and appreciated for their practical nature. Moreover, using these wipes may avoid the handling and transportation of bottles comprising lotions or milks.

Impregnated wipes may be wet or dry. Dry wipes can be moistened before use and may be impregnated, for example, with a foaming composition that generates lather when the wipe is moistened, as described, for example, in U.S. Pat. No. 4,303,543. Wet wipes may be impregnated with an aqueous composition, such as a makeup-removing lotion or a makeup-removing milk, for example, and they may be applied directly to the face or the body. Wet wipes may also be impregnated with an anhydrous composition comprising, for example, a mixture of oils and surfactants. The wipe may then be either used directly on the face or the body, or moistened beforehand with a small amount of water so as to emulsify the oils/surfactants mixture before application to the skin, as described, for example, in U.S. Pat. No. 6,136,775.

Patent Application No. FR 02/11607 filed on 19 Sep. 2002 discloses a cosmetic article, which may, for example, be in the form of a wipe or a glove, and which may be applied to moist skin, after a bath or a shower. Passing the glove over moist skin emulsifies the oil comprised in the glove and deposits it in the form of a soft and creamy moisturizing body milk. According to one example in the application, the glove comprises a highly absorbent nonwoven material, and it then may also have the advantage of allowing the skin to be simultaneously dried.

One problem posed by the implementation of the example described in the application arises from the fact that, despite the articles good water-absorbing capacity, such articles, once moistened, may have a tendency to release onto the skin, particles of the superabsorbent material that they contain, in response to this material being rubbed on the skin. These particles, engorged with water, may have a diameter of 1 mm or more. Their contact with the skin may result in an unpleasant sensation, which may be considered unacceptable by a great many users.

In addition, the quality of the wiping may be considered unsatisfactory, based upon the moist contact of the article with the skin when this article is engorged with water.

Thus, at least one of the objects disclosed herein is to produce a cosmetic process and a cosmetic article that solve at least some of the problems discussed above.

One embodiment disclosed herein is an article for the cosmetic treatment of moist skin or hair, which may also allow satisfactory wiping of the surface onto which the cosmetic treatment is applied.

Another embodiment is an article which improves at least one of the sensory qualities relating to the contact between the article and the surface onto which the treatment is applied, for example, the skin.

Other objects will emerge in the detailed description that follows.

At least some of these objects may be achieved by producing a cosmetic article comprising a support impregnated with a cosmetic composition, wherein the support comprising:
a) a contact structure, one face of which is configured so as to come into contact with a moistened surface onto which the cosmetic composition is applied, wherein the face is at least partly formed from at least one hydrophobic material, and
b) an absorbent structure adjacent to the contact structure, wherein the absorbent structure comprises at least one water-absorbing material such that the cosmetic article can absorb at least 10 times its weight of water, and
   wherein the contact structure is configured:
i) to be permeable to the water present on the moist surface, and
ii) to form a barrier to the at least one water-absorbing material, to prevent the at least one water-absorbing material, for example, when the at least one water-absorbing material is engorged with water, from coming into contact with the surface onto which the product is to be applied.

Thus, an article with high absorbency may be produced, which also comprises at least one good wiping property, while at the same time solving at least some of the problems discussed above. For example, the article disclosed herein may solve at least one of the problems relating to the unpleasant sensation resulting from the contact between the skin and the particles or fibers of the superabsorbent material that may become detached from the absorbent structure under the effect of the stresses resulting from the friction between the article and the surface to be treated.

For example, the contact structure may act as barrier and may keep the skin away from any water-engorged particle or fiber that might become detached from the absorbent structure.

In one embodiment, the barrier effect, with respect to the superabsorbent particles or fibers, may be obtained, as a function of the size characteristics of these particles or fibers, by varying, for example, the density and thickness of the contact structure. These properties may be chosen such that the spaces defined by the permeable structure may be sufficiently small compared with the size of the water-absorbent particles that might be released during the use of the article, so as to prevent them from passing through the contact structure and coming into contact with the skin.

The characteristic relating to the hydrophobic nature of the at least one hydrophobic material forming all or part of the face intended to come into contact with the surface to be treated may require the permeable contact structure to have a low hydrophilicity. This may promote the sensation of a dry contact between the skin and the cosmetic article. For example, the contact structure may be water-permeable insofar as it allows water to pass through but retains little or none of it, i.e. it does not absorb the water.

In one embodiment, the contact structure may comprise at least one nonwoven material whose weight per unit area ranges from 10 g/m² to 100 g/m² and, for example, from 15 g/m² to 60 g/m². The thickness of the contact structure may range from 0.3 mm to 1.5 mm and, for example, from 0.4 mm to 0.6 mm.

The absorption capacity of the article is measured according to the EDANA standard 10.4-02.

The moist surface onto which the cosmetic treatment is applied may for example, be chosen from at least one of skin, mucous membranes, hair, and scalp.

In one embodiment, the face of the contact structure, intended to come into contact with the skin, is at least partly formed from fibers comprising least one hydrophobic material, for example, chosen from polyethylene, polypropylene, and polyethylene terephthalate, which may have undergone a size treatment. The selection of appropriate fiber materials and the application of a sizing agent is well understood by the skilled artisan.

The article may be, for example, smooth and soft upon contact with the skin.

The contact structure may be produced using any art recognized method for forming a fibrous product. In one embodiment, the contact structure comprises a carded or extruded (“spunlaid”) and hot-bonded sized polypropylene or polyethylene terephthalate nonwoven material. The hot bonding may be obtained, for example, by calendering or by treating with hot air (“air-through bonded”).

Such contact structures are sold, for example, under the references PPTH 23 EIS at 23 g/m², PPTH 20 EIS at 20 g/m² and PPSS 15 EIS at 15 g/m² by the company Dounor, under the reference Comfort White Hydrophilic® at 10 g/m² by the company Fibertex, under the reference 111 018 HI at 18 g/m² by the company Fiberweb, or under the references Sawavlies® 34/00/61 and 34/00/53 at 20 g/m² and 22 g/m², respectively, by the company Sandler.

In another embodiment, the contact structure comprises a first face configured so as to come into contact with the surface to be treated, and a second face opposite the first, wherein the water permeability of the contact structure, in the direction from the first face to the second, is greater than the permeability of the structure in the opposite direction, wherein the absorbent structure is arranged on the second face side. This characteristic may limit the return of water from the absorbent structure to the contact structure, which may further improve the dry contact sensation produced by the cosmetic article disclosed herein.

In one embodiment, the first face may be formed from at least one water-bonded nonwoven material, for example, chosen from polyethylene and polypropylene, and the second face comprises at least one microperforated film, for example, polyethylene. The microperforated polyethylene film may be spunlaid or hot-bonded onto the nonwoven material. Such materials are sold, for example, by the company ACE SA (a subsidiary of the group Rheinsiche Kunstoff Werke AG).

The material used to produce the contact structure is also chosen so as to ensure the soft contact generally required by the application of a cosmetic product.

In one embodiment, the cosmetic article disclosed herein comprises at least one distribution layer configured, for example, by capillary action, so as to promote the distribution of the water passing through the water contact structure onto the absorbent structure, wherein the at least one distribution layer is either formed by the contact structure or formed by an additional structure located between the contact structure and the absorbent structure.

This characteristic may, for example, be advantageous for an application in which cosmetic treatment and wiping of the surface to be treated are combined, insofar as the latter function generally involves a translation movement over the moist skin. This then results in the front part of the article coming into contact with a more abundant amount of water (relative to the movement on the moist skin) than the rear part of the article. As a result, the impregnation of the absorbent structure is very unequal. The efficacy of the article is not optimum.

The at least one distribution layer may, for example, be a layer of low density, for example, chosen from hydrophobic materials, such as polypropylene and polyethylene terephthalate and may be chosen from nonwoven materials obtained by carding and thermal bonding (air-through bonded). This structure may allow the fibers, which are relatively airy, to convey the water by capillary action over the entire surface of the article and thus ensure good distribution of the water over the whole surface of the article. The impregnation of the absorbent structure with water is thereby found to be more uniform.

Examples of materials that may be used as the at least one distribution layer include nonwovens sold under the commercial references “dry web PROEF 12-012” (25 g/m² based on polypropylene and polyethylene terephthalate two-component fibres), “dry web TR2” (40 g/m² based on trilobal polyethylene terephthalate fibres) and “dry web T15 PROEF 15-029” (45 g/m² based on low-denier polyethylene terephthalate fibres), all sold by the company Libeltex. In one embodiment, Sawabond 4131 at 35 g/m² and May 1, 1951 at 45 g/m² sold by the company Sandler may be used.

In order to combine in a single structure, i.e. the contact web, the properties of softness on the skin and rapid and efficient distribution of water during the use of the article, bicomponent fibers may be used to promote the softness and also the weldability (for example, when it is desired to produce a structure in the form of a glove) and coarse-denier fibers may be used to promote rapid passage of water to the absorbent structure. A small proportion of viscose fibers may also be added to promote good drainage. Appropriate fibers and structures would be readily selectable by the skilled artisan.

For example, the at least one water-absorbing material may be chosen from powders, such as sodium glycolate of crosslinked starches, starches grafted with sodium polyacrylate, crosslinked acrylic acid/sodium acrylate copolymer, for example, Primogel®, sold by the company Avebe, and Sanwet® sold by the company BASF.

The powder may be deposited inside a structure formed from at least one water-permeable layer. In one embodiment, the structure may be a nonwoven material comprising a single layer of fibers needled in the presence of superabsorbent powder.

For example, the superabsorbent powder may be incorporated via an electrostatic process into a nonwoven material that is already formed, for example by carding and then consolidation via hydro-bonding or needling.

In another embodiment, the nonwoven material comprises two superposed sheets delimiting between them at least one pocket comprising the powder. For example, the nonwoven material may comprise a single pocket between the two sheets or a plurality of pockets distributed over the whole surface of the absorbent structure.

Examples of nonwovens that may be used to produce the absorbent structure that may be used include those sold under the names Dritex 120NN42 and 130WNNF60 by the company Georgia Pacific, and those sold under the names HY0201040, HY0101046, HY0101100 and HY0301038 by the company BBA.

The powders may be mixed with fibers, for example, cellulose fibers derived, for example, from paper pulp. Such absorbent structures comprising a powder as a mixture with fibers are often referred to by the term “fluff” (material comprising a mixture of superabsorbent powder and cellulose fibers without final bonding treatment). As a result of its structure, the “fluff” lacks strength and it may be held between two layers of nonwoven material. Such products are sold, for example, by the companies Georgia Pacific (USA), Tembec and Tartas (France).

According to another embodiment, the at least one water-absorbing material may be chosen from fibers. Such fibers may be carded and needle-bonded. Examples of the fibers that may be used include:
i) fibers of an acrylate copolymer, for example, the nonwovens sold by the company Oasis under the references 2130 (200 g/m², 50% superabsorbent fibres/50% PET fibers carded and needled), 2160 (145 g/m², 30% superabsorbent fibres/70% PET fibers carded and needled) and 2091 (30% superabsorbent fibers/70% pulp and airlaid and hot-bonded bicomponent fibers), and
ii) carboxymethyl cellulose and alginate fibers, both sold by the company Acordis.

In one embodiment, the absorbent structure is configured such that the article can absorb at least 15 times its weight of water and, for example, at least 20 times its weight of water. In another embodiment, the article may be able to absorb greater than 50 g of water.

The cosmetic composition used in the article disclosed herein may be chosen from compositions for treating the skin, mucous membranes, the hair and/or the scalp.

In one embodiment, the article is essentially anhydrous. As used herein, the term “essentially anhydrous” means comprises no water, comprises no more than 10% by weight of water and, further, comprises no more than 5% by weight, of water. The amount of water in the article may thus range from 0 to 10% by weight, and, for example, from 0 to 5% by weight, relative to the total weight of the article.

The cosmetic composition may comprise at least one agent chosen from self-tanning agents, slimming agents, sunscreens, moisturizers, heating agents, hair disentangling agents, skin and hair conditioners, keratolytic agents, skin-lightening agents, for example, depigmenting agents, and hair-colouring agents. The article may be used on the entire body or the hair, but may also be used as a local treatment.

In one embodiment, the cosmetic composition may be liquid, i.e. it may have a viscosity of less than 150 mPa.s and, further, for example, less than 100 mPa.s. The viscosity may, for example, range from 1 mPa.s to 100 mPa.s, measured at room temperature (about 25° C.) using a Rheomat RM 180 machine, with a No. 1 or 2 spindle depending on the viscosity of the liquid. In some embodiments, the cosmetic composition may be in the form of a powder.

The article may have any size and form that is suitable for the desired end use. It may thus, for example, be provided in forms chosen from wipes and small towels, for example, of substantially rectangular shape, gloves and mittens, which is easy to slip onto the hand, and a compress. The compress may have an area ranging, for example, from 0.005 m² to 0.1 m² and, further, for example, from 0.01 m² to 0.07 m².

The degree of impregnation of the composition on the support may range, for example, from 10% to 1500%, further, for example, from 50% to 500% and, even further, for example, from 70% to 250%. The techniques for impregnating substrates with compositions are well known in this field and are all applicable to the present disclosure. For example, the impregnation composition may be added to the substrate via at least one technique chosen from immersion, coating, vaporization, electrostatic deposition, and dusting (for example, before joining the absorbent structure to the contact structure).

In one embodiment, on the side opposite the contact structure, the absorbent structure may comprise at least one application contact layer configured so as to come into contact with a hand manipulating the article, wherein the at least one application contact layer comprises at least one material chosen from polypropylene, viscose, and polyethylene terephthalate. In another embodiment, the at least one application contact layer may comprise at least one nonwoven material chosen from carded hydrophobic polypropylene fibers and filaments deposited as a uniform layer via the “spunlaid” technology and then hot-bonded. For this layer, a dry contact and a pleasant feel may generally be desired.

The at least one application contact layer of the absorbent structure may also stop the diffusion of the liquid through the at least one absorbent layer in the case where the at least one absorbent layer does not manage to contain all the water collected. The at least one contact layer may also serve to prevent any loss of powder or of fibres from the at least one absorbent layer during use.

For examples, products that may be mentioned include the commercial references PPSMS/17/E/O/S and PPSS/17/E/O/S at 17 g/m² sold by the company Dounor, the commercial reference Comfort White Hydrophobic® at 15 g/m² sold by the company Fibertex, the reference 197 DIMSET020HOB at 20 g/m² sold by the company Fiberweb, and the reference Sawabond® 4111 at 22 g/m² sold by the company Sandler.

In one embodiment, in order to make the article reversible, i.e. to be able to use both sides, it may be envisaged to use the same contact structure on each side of the absorbent structure. It may be advantageous, for example, to follow this option in the case where the article is configured in the form of a towel or a wipe.

Another embodiment relates to a cosmetic process for treating the skin, mucous membranes, the hair and/or the scalp comprising:
a) wetting the surface to be treated; and
b) wiping the surface using the cosmetic article disclosed herein.

As used herein, the term “cosmetic treatment” means a treatment performed with at least one cosmetic product. The term “cosmetic product” means a product as defined in Directive 93/35/EEC of the Council of 13 Jun. 1993.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed composition. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contain certain errors necessarily resulting from the standard deviation found in their respective testing measurements. The following examples are intended to illustrate the present disclosure without limiting the scope as a result.

Example of a Bodycare Glove:

The composition used to perform this example is described below:

| Oils | |
|---|---|
| Ethylhexyl palmitate | 25 |
| Parleam oil | 20 |
| Musk rose oil | 5 |
| Surfactants | |
| PEG-20 glyceryl triisostearate | 10 |
| Active agents | |
| Glycerol | 40 |

The composition according to this example was prepared according to the following process:

The mixture of the oils were first prepared and then the surfactants were incorporated therein at room temperature or at elevated temperature, depending on whether they are in liquid or solid form. The hydrophilic gelling agent and the adjuvants were then incorporated into the mixture obtained.

The degree of impregnation of the composition onto the article was 200%. The article was configured in the form of a glove.

The glove consisted of two identical multilayer structures formed from nonwovens, the structures being cut into the desired shape and welded, bonded or sewn at the periphery.

On use, after a shower or bath, the glove was passed over the moist skin, over the entire body. Passing the glove over the moist skin emulsified the oil contained in the glove and deposited it in the form of a soft and creamy moisturizing body milk.

Example of a Hair-Conditioning Towel

The composition contained a mixture of cetylstearyl alcohol and of N,N-distearoyloxyethyl-N-methyl-N-hydroxyethylammonium methosulfate, 25/75.

The product was incorporated into the article either in pure form as a powder, or in a form prediluted in water, and the article was then totally or partially dried so as to have an article that was substantially dry to the touch.

Example of a Body Moisturizing Towel

The composition comprised:
Glycerol: qs 100
Aloe vera: 0.01%
Menthol: 0.2%
Extract of witch hazel (at 1% in water): 1%.

The ingredients were mixed together at room temperature. The degree of impregnation of the composition onto the article is 200%. The article was configured in the form of a small towel or a glove.

Example of a Self-Tanning Glove

The composition used according to this example comprised:
β,β′-camphorsulfonic acid [1,4-divinylbenzene] as an aqueous 33% solution: 6%
Sodium hydroxide: 0.28%
Glycerol: 70.72%
Water: 5%
Trialkyl citrate: 8%
Dihydroxyacetone: 10%.

Its mode of preparation was identical to that of the preceding example.

The table below describes various stacks of layers that can form either the article directly in the case of a wipe or a towel, or at least one of the faces of the article when it is configured in the form of a glove.

| 1 | 2 | 3 | 4 | 5 | 6 | 7 | 8 | 9 | 10 | 11 | 12 | 13 | 14 |
|---|---|---|---|---|---|---|---|---|---|---|---|---|---|
| A1 | A1 | A1 | A1 | A1 | A1 | A1 | A1 | A1 | A1 | A2 | A2 | A2 | A2 |
| | | | B2 | B2 | B2 | | | B2 | B2 | | B2 | B3 | B4 |
| C2 | C3 | C4 | C2 | C4 | C4 | C3 | C4 | C3 | C4 | C3 | C3 | C3 | C3 |
| D2 | D2 | D2 | D2 | D2 | D2 | D1 | D1 | D1 | D1 | D2 | D2 | D2 | D2 |

A1-A2 denote the contact structure of the article.

B2-B4 denote the distribution layer present in some of the embodiments listed above.

C2-C4 denote the absorbent structure.

D1-D2 denote the layer intended to be in contact with the hand.

Commercial/technical references corresponding to each of the letters A1-A2, B2-B4, C2-C4 and D1-D2 are illustrated below.
A1: PPTH20EIS at 20 g/m² from Dounor
A2: PPSS15EIS at 15 g/m² from Dounor
B2: Dry web T15PROEF 15-029″ at 45 g/m² from Libeltex
B3: Dry web PROEF 12-012″ at 25 g/m² from Libeltex
B4: Dry web TR2″ at 40 g/m² from Libeltex
C2: Reference 2160 at 145 g/m² from Oasis
C3: Needled carboxymethyl cellulose at 90 g/m²
C4: Needled alginate at 150 g/m²
D1: PPSMS/17/E/O/S at 17 g/m² from Dounor
D2: PPSS/17/E/O/S at 17 g/m² from Dounor.

1. A cosmetic article comprising a support impregnated with a cosmetic composition, wherein the support comprises:
a) a contact structure, one face of which is configured so as to come into contact with a moistened surface onto which the cosmetic composition is applied, wherein the face is at least partly formed from at least one hydrophobic material, and
b) an absorbent structure adjacent to the contact structure, wherein the absorbent structure comprises at least one water-absorbing material such that the cosmetic article can absorb at least 10 times its weight of water,
wherein the contact structure is configured:
i) to be permeable to the water present on the moist surface, and
ii) to form a barrier to the at least one water-absorbing material so as to prevent the at least one water-absorbing material from coming into contact with the surface onto which the cosmetic composition is applied.

2. The cosmetic article according to claim 1, wherein the contact structure is configured so as to form a barrier to the at least one water-absorbing material, to prevent the at least one water-absorbing material, when the at least one water-absorbing material is engorged with water, from coming into contact with the surface onto which the cosmetic composition is applied.

3. The cosmetic article according to claim 1, wherein the at least one contact structure is at least partly formed from fibers comprising at least one hydrophobic material.

4. The cosmetic article according to claim 3, wherein the at least one hydrophobic material is chosen from polyethylene, polypropylene, and polyethylene terephthalate.

5. The cosmetic article according to claim 1, wherein the contact structure:
i) comprises at least one nonwoven material having a weight per unit area ranging from 10 g/m² to 100 g/m²; and
ii) has a thickness ranging from 0.3 mm to 1.5 mm.

6. The cosmetic article according to claim 5, wherein the at least one nonwoven material has a weight per unit area ranging 15 g/m² to 60 g/m².

7. The cosmetic article according to claim 5, wherein the contact structure has a thickness ranging from 0.4 mm to 0.6 mm.

8. The cosmetic article according to claim 1, wherein the contact structure comprises a first face and a second face opposite the first face, wherein the water permeability of the contact structure, in the direction from the first face to the second face, is greater than the permeability of the contact structure in the opposite direction, and wherein the at least one absorbent structure is arranged on the second face side.

9. The cosmetic article according to claim 8, wherein the first face comprises at least one nonwoven material and the second face comprises at least one microperforated film.

10. The cosmetic article according to claim 9, wherein the at least one nonwoven material is chosen from polyethylene and polypropylene.

11. The cosmetic article according to claim 9, wherein the at least one microperforated film is polyethylene.

12. The cosmetic article according to claim 1, further comprising at least one distribution layer capable of promoting the distribution of the water passing through the contact structure on the at least one absorbent structure, wherein the at least one distribution layer is either formed by the contact structure or formed by an additional structure located between the contact structure and the absorbent structure.

13. The cosmetic article according to claim 12, wherein the at least one distribution layer is capable via capillary action to promote the distribution of water.

14. The cosmetic article according to claim 12, wherein the at least one distribution layer comprises at least one hydrophobic material.

15. The cosmetic article according to claim 14, wherein the at least one hydrophobic material is chosen from polypropylene and polyethylene terephthalate.

16. The cosmetic article according to claim 1, wherein the at least one water-absorbing material is chosen from powders.

17. The cosmetic article according to claim 16, wherein the powders are chosen from sodium glycolate of crosslinked starches, starches grafted with sodium polyacrylate, and crosslinked acrylic acid/sodium acrylate copolymers.

18. The cosmetic article according to claim 16, wherein the powders are arranged inside a structure formed from at least one water-permeable layer.

19. The cosmetic article according to claim 18, wherein the structure comprises a single water-permeable layer comprising at least one nonwoven material comprising the powders or two superposed water-permeable layers delimiting between them at least one pocket comprising the powders.

20. The cosmetic article according to claim 18, wherein the powders are in a mixture with fibers.

21. The cosmetic article according to claim 20, wherein the fibers are chosen from cellulose fibers.

22. The cosmetic article according to claim 1, wherein the at least one water-absorbing material is chosen from fibers.

23. The cosmetic article according to claim 22, wherein the fibers are chosen from acrylate copolymers, carboxymethyl cellulose, and alginate.

24. The cosmetic article according to claim 1, wherein the absorbent structure is configured so that the article can absorb at least 15 times its weight of water.

25. The cosmetic article according to claim 24, wherein the absorbent structure is configured so that the article can absorb at least 20 times its weight of water.

26. The cosmetic article according to claim 1, wherein the article is capable of absorbing greater than 50 g of water.

27. The cosmetic article according to claim 1, wherein the composition chosen from compositions for treating the skin, mucous membranes, the hair, and/or the scalp.

28. The cosmetic article according to claim 1, wherein the article is essentially anhydrous.

29. The cosmetic article according to claim 1, wherein the composition comprises at least one agent chosen from sunscreens, self-tanning agents, slimming agents, moisturizers, heating agents, hair disentangling agents, skin and hair conditioners, keratolytic agents, skin-lightening agents, and hair-coloring agents.

30. The cosmetic article according to claim 29, wherein the skin-lightening agents are chosen from depigmenting agents.

31. The cosmetic article according to claim 1, wherein, on the side opposite the contact structure, the absorbent structure is adjacent to at least one contact layer configured so as to be in contact with a hand manipulating the article.

32. The cosmetic article according to claim 31, wherein the at least one contact layer comprises at least one material chosen from polypropylene, viscose, and polyethylene terephthalate.

33. The cosmetic article according to claim 1, wherein the impregnation of the article with the cosmetic composition is performed by at least one technique chosen from spraying, immersion, dusting, coating, and electrostatic techniques.

34. The cosmetic article according to claim 1, wherein the degree of impregnation of the composition on the support ranges from 10% to 1500%.

35. The cosmetic article according to claim 34, wherein the degree of impregnation of the composition on the support ranges from 50% to 500%.

36. The cosmetic article according to claim 35, wherein the degree of impregnation of the composition on the support ranges from 70% to 250%.

37. The cosmetic article according to claim 1, wherein the article is provided in a form chosen from a glove, a mitten, a towel, a wipe, and a compress.

38. The cosmetic article according to claim 37, wherein the wipes and the towels are of a substantially rectangular shape.

39. The cosmetic article according to claim 1, wherein the contact structure is adjacent to both the first face and the second face of the absorbent structure.

40. A cosmetic process for treating the skin, mucous membranes, the hair and/or the scalp comprising,
a) wetting the surface to be treated, and
b) wiping the surface using at least one cosmetic article, comprising a support impregnated with a cosmetic composition, wherein the support comprises:
a) a contact structure, one face of which is configured so as to come into contact with a moistened surface onto which the cosmetic composition is applied, wherein the face is at least partly formed from at least one hydrophobic material, and
b) an absorbent structure adjacent to the contact structure, wherein the absorbent structure comprises at least one water-absorbing material such that the cosmetic article can absorb at least 10 times its weight of water,
wherein the contact structure is configured:
i) to be permeable to the water present on the moist surface, and
ii) to form a barrier to the at least one water-absorbing material so as to prevent the at least one water-absorbing material from coming into contact with the surface onto which the cosmetic composition is applied.
